(19) United States
(12) Patent Application Publication
(10) Pub. No.: US 2017/0259039 A1
(43) Pub. Date: Sep. 14, 2017

(54) SUBCUTANEOUS TUNNELING DEVICE WITH ELONGATED OPEN SLOT SHEATH

(71) Applicant: Cardiac Pacemakers, Inc., St. Paul, MN (US)

(72) Inventor: Jack Gordon, Minneapolis, MN (US)

(21) Appl. No.: 15/457,766

(22) Filed: Mar. 13, 2017

Related U.S. Application Data
(60) Provisional application No. 62/307,944, filed on Mar. 14, 2016.

(51) Int. Cl. A61M 25/06; A61M 25/01; A61M 25/00

(52) U.S. Cl. CPC A61M 25/0668; A61M 25/0068; A61M 25/0097; A61M 25/0194; A61M 2025/0006; A61M 2205/583; A61M 2205/582; A61M 2025/0681

(57) ABSTRACT

An introducer sheath includes an elongated outer sheath and an elongated inner sheath coaxially disposed within the outer sheath. The elongated outer sheath includes an outer sheath wall extending circumferentially about a longitudinal axis of the outer sheath to an extent greater than 180° and less than 360° to define an outer sheath slot. The elongated inner sheath includes an inner sheath wall extending circumferentially about a longitudinal axis of the inner sheath to an extent greater than the difference between 360° and the circumferential extent of the outer sheath wall, and less than 360° to define an inner sheath slot. The inner sheath is rotatable relative to the outer sheath to form an enclosed lumen when the outer and inner sheath slots are not aligned, and to form an open slot along the entire length of the introducer sheath when the outer and inner sheath slots are aligned.

### CROSS-REFERENCE TO RELATED APPLICATION

This application claims priority to Provisional Application No. 62/307,944, filed Mar. 14, 2016, which is herein incorporated by reference in its entirety.

### TECHNICAL FIELD

The present invention relates to medical devices and methods for implanting medical devices. More specifically, the invention relates to devices and methods for implanting a lead or a catheter by tunneling.

### BACKGROUND

Medical devices, such as electrical leads and catheters, may be subcutaneously implanted within a patient through the use of a tunneling tool. The tunneling tool typically includes an elongated tunneling rod or stylet inserted through a lumen of a splitable or peelable introducer sheath. The tip of the tunneling rod protrudes through the end of the introducer sheath as the tunneling rod and introducer sheath are inserted through an incision in a patient. The tapered tip of the tunneling rod eases the transit of the tunneling tool as the tunneling rod is driven to the desired location under the patient's skin. Once the tip of the tunneling rod is at the desired implant location, the tunneling rod is removed and the introducer sheath is left behind to provide a passageway for insertion of the lead or catheter through lumen of the introducer sheath to the implant location. Following insertion of the lead or catheter, the introducer sheath is extracted from the patient.

### SUMMARY

Example 1 is an introducer sheath including an elongated outer sheath and an elongated inner sheath. The elongated outer sheath includes a proximal end, a distal end, and an outer sheath wall extending from the proximal end to the distal end. The outer sheath wall extends circumferentially about a longitudinal axis of the outer sheath to an extent greater than 180° and less than 360° to define an outer sheath slot extending from the proximal end through the distal end. The elongated inner sheath includes a proximal end, a distal end, and an inner sheath wall extending from the proximal end to the distal end. The inner sheath wall extends circumferentially about a longitudinal axis of the inner sheath to an extent greater than the difference between 360° and the circumferential extent of the outer sheath wall, and less than 360° to define an inner sheath slot extending from the proximal end through the distal end. The inner sheath is coaxially disposed within the outer sheath. The inner sheath is rotatable relative to the outer sheath and is configured to form an enclosed lumen when the outer sheath slot is not aligned with the inner sheath slot, and to form an open slot along the entire length of the introducer sheath when the outer sheath slot is aligned with the inner sheath slot.

In Example 2, the introducer sheath of example 1, wherein the outer sheath slot is parallel to the outer sheath longitudinal axis and the inner sheath slot is parallel to the inner sheath longitudinal axis.

In Example 3, the introducer sheath either of examples 1 or 2, wherein the outer sheath wall extends circumferentially about the longitudinal axis of the outer sheath to an extent of at least 190° to no more than 200°, and the inner sheath wall extends circumferentially about the longitudinal axis of the inner sheath to an extent of at least 190° to no more than 200°.

In Example 4, the introducer sheath of any of examples 1-3, wherein a width of the inner sheath slot in a direction perpendicular to the longitudinal axis of the inner sheath ranges from about 0.125 inches (3.18 mm) to about 0.5 inches (12.7 mm).

In Example 5, the introducer sheath of any of examples 1-4, wherein the distal ends of each of the outer sheath wall and the inner sheath wall include a chamfer.

In Example 6, the introducer sheath of any of examples 1-5, further including an outer sheath handle and an inner sheath handle. The outer sheath handle is connected to the proximal end of the outer sheath. The outer sheath handle includes an outer sheath handle slot aligned with, and at least as wide as, the outer sheath slot. The inner sheath handle is connected to the proximal end of the inner sheath. The inner sheath handle includes an inner sheath handle slot aligned with, and at least as wide as, the inner sheath slot. The inner sheath handle is rotatable relative to the outer sheath handle to rotate the inner sheath relative to the outer sheath.

In Example 7, the introducer sheath of example 6, further including a locking device configured selectively lock the outer sheath handle to the inner sheath handle to selectively lock rotational positions of the outer sheath and the inner sheath with respect to each other.

In Example 8, the introducer sheath of either of examples 6 or 7, wherein at least one of the outer sheath handle and the inner sheath handle include a rotational stop configured to prevent further rotation of the inner sheath relative to the outer sheath once the outer and inner sheath slots are aligned with each other.

In Example 9, the introducer sheath of any of examples 6-8, wherein the outer sheath handle and the inner sheath handle each include surface features configured to provide at least one of a visual indication and a tactile indication once the outer and inner sheath slots are aligned with each other.

Example 10 is a tunneling device for implanting a lead or catheter. The device includes an introducer sheath according to any of examples 6-9 and a tunneling rod. The tunneling rod is removably inserted into the lumen of the introducer sheath. The tunneling rod extends from a proximal end to a distal end. The distal end includes a tapered tip. The tapered tip projects at least partially beyond the distal ends of the outer sheath and the inner sheath and defines a distal end of the tunneling device.

In Example 11, the tunneling device of example 10, wherein the tunneling rod further includes a rod handle connected to the proximal end of the tunneling rod, the rod handle proximal to the inner sheath handle.

In Example 12, the tunneling device of either of examples 10 or 11, wherein the tunneling rod is curved along at least a portion of its length and the introducer sheath is formed of a material that is flexible and resilient.

Example 13 is a method for implanting a lead or catheter within a patient. The method includes making an incision at a location of implantation, inserting into the incision a distal end of a tunneling device including a tunneling rod and an introducer sheath according to any of examples 10-12, driving the distal end of the tunneling device to a location corresponding to the desired placement of the lead or catheter, removing the tunneling rod from a proximal end of the introducer sheath, inserting the lead or catheter into the introducer sheath until the lead or catheter projects beyond a distal end of the introducer sheath, rotating the inner sheath relative to the outer sheath until the inner sheath slot and the outer sheath slot are aligned to each other to form an open slot along the entire length of the introducer sheath, and withdrawing the introducer sheath from the patient, wherein the implanted lead or catheter passes through the open slot.

In Example 14, the method of example 13, wherein the introducer sheath includes an outer sheath handle connected to the proximal end of the outer sheath, the outer sheath handle including an outer sheath handle slot aligned with, and at least as wide as, the outer sheath slot; and an inner sheath handle connected to the proximal end of the inner sheath, the inner sheath handle including an inner sheath handle slot aligned with, and at least as wide as, the inner sheath slot, wherein the inner sheath handle is rotatable relative to the outer sheath handle to rotate the inner sheath relative to the outer sheath, wherein rotating the inner sheath relative to the outer sheath includes unlocking the outer sheath handle from the inner sheath handle, rotating the inner sheath handle relative to the outer sheath handle until a rotational stop is reached, the rotational stop corresponding to the alignment of the inner sheath slot and the outer sheath slot to each other, and locking the outer sheath handle to the inner sheath handle.

In Example 15, the method of example 14, wherein the outer sheath handle and the inner sheath handle each include surface features configured to provide at least one of a visual indication and a tactile indication once the outer and inner sheath slots are aligned with each other, the method further including verifying alignment of the outer and inner sheath slots with each other by at least one of visual and tactile indication of the surface features before withdrawing the introducer sheath from the patient.

In Example 16 an introducer sheath including an elongated outer sheath and an elongated inner sheath. The elongated outer sheath includes a proximal end, a distal end, and an outer sheath wall extending from the proximal end to the distal end. The outer sheath wall extends circumferentially about a longitudinal axis of the outer sheath to an extent greater than 180° and less than 360° to define an outer sheath slot extending from the proximal end through the distal end. The outer sheath slot is parallel to the longitudinal axis. The elongated inner sheath includes a proximal end, a distal end, and an inner sheath wall extending from the proximal end to the distal end. The inner sheath wall extends circumferentially about a longitudinal axis of the inner sheath to an extent greater than the difference between 360° and the circumferential extent of the outer sheath wall, and less than 360° to define an inner sheath slot extending from the proximal end through the distal end. The inner sheath slot is parallel to the longitudinal axis. The inner sheath is coaxially disposed within the outer sheath. The inner sheath rotatable relative to the outer sheath and configured to form an enclosed lumen when the outer sheath slot is not aligned with the inner sheath slot, and to form an open slot along the entire length of the introducer sheath when the outer sheath slot is aligned with the inner sheath slot.

In Example 17, the introducer sheath of example 16, wherein the outer sheath wall extends circumferentially about the longitudinal axis of the outer sheath to an extent of at least 190° to no more than 200°, and the inner sheath wall extends circumferentially about the longitudinal axis of the inner sheath to an extent of at least 190° to no more than 200°.

In Example 18, the introducer sheath of either of examples 16 or 17, wherein a width of the inner sheath slot in a direction perpendicular to the longitudinal axis of the inner sheath ranges from about 0.125 inches (3.18 mm) to about 0.5 inches (12.7 mm).

In Example 19, the introducer sheath of any of examples 16-18, wherein the distal ends of each of the outer sheath wall and the inner sheath wall include a chamfer.

In Example 20, the introducer sheath of any of examples 16-19, further including an outer sheath handle and an inner sheath handle. The outer sheath handle is connected to the proximal end of the outer sheath. The outer sheath handle includes an outer sheath handle slot aligned with, and at least as wide as, the outer sheath slot. The inner sheath handle is connected to the proximal end of the inner sheath. The inner sheath handle includes an inner sheath handle slot aligned with, and at least as wide as, the inner sheath slot. The inner sheath handle is rotatable relative to the outer sheath handle to rotate the inner sheath relative to the outer sheath.

In Example 21, the introducer sheath of example 20, further including a locking device configured selectively lock the outer sheath handle to the inner sheath handle to selectively lock rotational positions of the outer sheath and the inner sheath with respect to each other.

In Example 22, the introducer sheath of example 21, wherein the locking device is a set screw.

In Example 23, the introducer sheath of any of examples 20-22, wherein at least one of the outer sheath handle and the inner sheath handle include a rotational stop configured to prevent further rotation of the inner sheath relative to the outer sheath once the outer and inner sheath slots are aligned with each other.

In Example 24, the introducer sheath of any of examples 20-23, wherein the outer sheath handle and the inner sheath handle each include surface features configured to provide at least one of a visual indication and a tactile indication once the outer and inner sheath slots are aligned with each other.

Example 25 is a tunneling device for implanting a lead or catheter. The device includes an introducer sheath and a tunneling rod. The introducer sheath includes an elongated outer sheath and an elongated inner sheath. The elongated outer sheath includes a proximal end, a distal end, and an outer sheath wall extending from the proximal end to the distal end. The outer sheath wall extends circumferentially about a longitudinal axis of the outer sheath to an extent greater than 180° and less than 360° to define an outer sheath slot extending from the proximal end through the distal end. The outer sheath slot is parallel to the longitudinal axis. The elongated inner sheath includes a proximal end, a distal end, and an inner sheath wall extending from the proximal end to the distal end. The inner sheath wall extends circumferentially about a longitudinal axis of the inner sheath to an extent greater than the difference between 360° and the circumferential extent of the outer sheath wall, and less than 360° to define an inner sheath slot extending from the proximal end through the distal end. The inner sheath slot is parallel to the longitudinal axis. The inner sheath is coaxially disposed within the outer sheath. The inner sheath rotatable relative to the outer sheath and configured to form an enclosed lumen when the outer sheath slot is not aligned with the inner sheath slot, and to form an open slot along the entire length of the introducer sheath when the outer sheath slot is aligned with the inner sheath slot. The tunneling rod is removably inserted into the lumen of the introducer sheath. The tunneling rod extends from a proximal end to a distal end. The distal end includes a tapered tip. The tapered tip projects at least partially beyond the distal ends of the outer sheath and the inner sheath and defines a distal end of the tunneling device.

In Example 26, the tunneling device of example 25, wherein the tunneling rod is curved along at least a portion of its length and the introducer sheath is formed of a material that is flexible and resilient.

In Example 27, the tunneling device of either of examples 25 or 26, wherein the introducer sheath further includes an outer sheath handle and an inner sheath handle, and the tunneling rod further includes a rod handle. The outer sheath handle is connected to the proximal end of the outer sheath. The outer sheath handle includes an outer sheath handle slot aligned with, and at least as wide as, the outer sheath slot. The inner sheath handle is connected to the proximal end of the inner sheath. The inner sheath handle includes an inner sheath handle slot aligned with, and at least as wide as, the inner sheath slot. The inner sheath handle is rotatable relative to the outer sheath handle to rotate the inner sheath relative to the outer sheath. The tunneling rod further includes a rod handle connected to the proximal end of the tunneling rod The rod handle is proximal to the inner sheath handle.

In Example 28, the tunneling device of any of examples 25-27, wherein the introducer sheath further includes a locking device configured selectively lock the outer sheath handle to the inner sheath handle to selectively lock rotational positions of the outer sheath and the inner sheath with respect to each other.

In Example 29, the tunneling device of example 28, wherein the locking device is a set screw.

In Example 30, the tunneling device of any of examples 25-29, wherein at least one of the outer sheath handle and the inner sheath handle include a rotational stop configured to prevent further rotation of the inner sheath relative to the outer sheath once the outer and inner sheath slots are aligned with each other.

In Example 31, the tunneling device of any of examples 25-30, wherein the outer sheath handle and the inner sheath handle each include surface features configured to provide at least one of a visual indication and a tactile indication once the outer and inner sheath slots are aligned with each other.

In Example 32, the tunneling device of any of examples 27-31, wherein the tunneling rod further includes a locking device configured to selectively lock the rod handle to at last one of the outer sheath handle and the inner sheath handle.

Example 33 is a method for implanting a lead or catheter within a patient. The method includes making an incision at a location of implantation and then inserting into the incision a distal end of a tunneling device including a tunneling rod and an introducer sheath. The introducer sheath includes an outer sheath coaxially aligned with an inner sheath. The outer sheath includes an outer sheath slot extending the length of the outer sheath. The inner sheath includes an inner sheath slot extending the length of the inner sheath. The inner sheath slot not aligned with the outer sheath slot. The method further includes driving the distal end of the tunneling device to a location corresponding to the desired placement of the lead or catheter, removing the tunneling rod from a proximal end of the introducer sheath, inserting the lead or catheter into the introducer sheath until the lead or catheter projects beyond a distal end of the introducer sheath, rotating the inner sheath relative to the outer sheath until the inner sheath slot and the outer sheath slot are aligned to each other to form an open slot along the entire length of the introducer sheath, and withdrawing the introducer sheath from the patient, wherein the implanted lead or catheter passes through the open slot.

In Example 34, the method of example 33, wherein the introducer sheath includes an outer sheath handle and an inner sheath handle. The outer sheath handle is connected to a proximal end of the outer sheath. The outer sheath handle includes an outer sheath handle slot aligned with, and at least as wide as, the outer sheath slot. The inner sheath handle is connected to a proximal end of the inner sheath. The inner sheath handle includes an inner sheath handle slot aligned with, and at least as wide as, the inner sheath slot, wherein the inner sheath handle is rotatable relative to the outer sheath handle to rotate the inner sheath relative to the outer sheath, wherein rotating the inner sheath relative to the outer sheath includes unlocking the outer sheath handle from the inner sheath handle, rotating the inner sheath handle relative to the outer sheath handle until a rotational stop is reached, the rotational stop corresponding to the alignment of the inner sheath slot and the outer sheath slot to each other, and locking the outer sheath handle to the inner sheath handle.

In Example 35, the method of example 34, wherein the outer sheath handle and the inner sheath handle each include surface features configured to provide at least one of a visual indication and a tactile indication once the outer and inner sheath slots are aligned with each other, the method further including verifying alignment of the outer and inner sheath slots with each other by at least one of visual and tactile indication of the surface features before withdrawing the introducer sheath from the patient.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIGS. 1A and 1B are perspective views of an exemplary tunneling device.

FIGS. 2A-2B are perspective views of a portion of an exemplary introducer sheath showing an enclosed-lumen configuration and an open-lumen configuration, respectively.

FIGS. 3A-3B are schematic cross-sections of the exemplary introducer sheath showing the enclosed-lumen configuration and the open-lumen configuration of FIGS. 2A and 2B, respectively.

FIGS. 4A and 4B illustrate steps of implanting a lead or catheter within a patient in accordance with disclosed embodiments.

FIGS. 5A and 5B are perspective views of a portion of an exemplary introducer sheath illustrating additional steps of implanting the lead or catheter within the patient in accordance with disclosed embodiments.

FIGS. 6A-6C are schematic cross-sections of the exemplary introducer sheath further illustrating the additional steps of implanting the lead or catheter within the patient in accordance with disclosed embodiments.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

In some cases, insertion of a lead or catheter through an introducer sheath as described above occurs under conditions requiring connection of a system or device to the lead or catheter. For example, implantation of a lead for a subcutaneous implantable cardioverter defibrillator (S-ICD) may be done while the lead is connected to the cardioverter defibrillator. In such cases, the introducer sheath must be longitudinally split or peeled apart so that it can be extracted from the patient without the need to disconnect the cardioverter defibrillator. Although such introducer sheaths are designed to be split apart, the process requires the surgeon to use both hands to apply a significant force to break apart a hub of the introducer sheath, and then to use both hands to split and extract the sheath at the same time. Such effort may result in a left or right handed shift in the sheath, causing a shift in the position of the lead or catheter from its intended implant position. The lead or catheter may also be undesirably rotated due to torque influences during the process of splitting and extracting the introducer sheath.

Embodiments described below include an introducer sheath including coaxial outer and inner sheaths. The coaxial sheaths are rotatable relative to each other. Each of the coaxial sheaths has an open longitudinal slot though a wall of the sheath and running the full length of the sheath. When the sheaths are oriented such that the slots do not align, the sheaths form an enclosed sheath suitable for use as an introducer sheath. When the sheaths are oriented such that the openings align, they form an open slot extending the full length of the introducer sheath. The open slot is wide enough to permit the lead or catheter to pass out of the introducer sheath through the open slot. Rotation of the sheaths to align the slots requires less force than breaking the hub of a splittable introducer, resulting in little, if any, left or right handed shift in the sheath. Once the sheaths are rotated to form the open slot, the introducer may simply be backed out of the patient, producing little, if any, side load or rotational forces to disturb the placement of the lead or catheter.

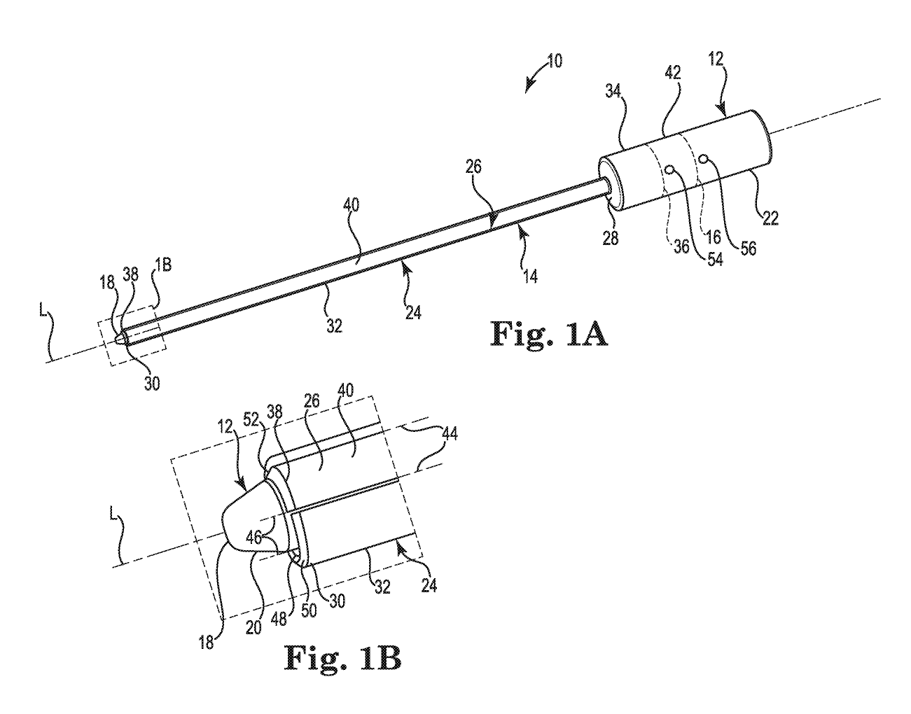

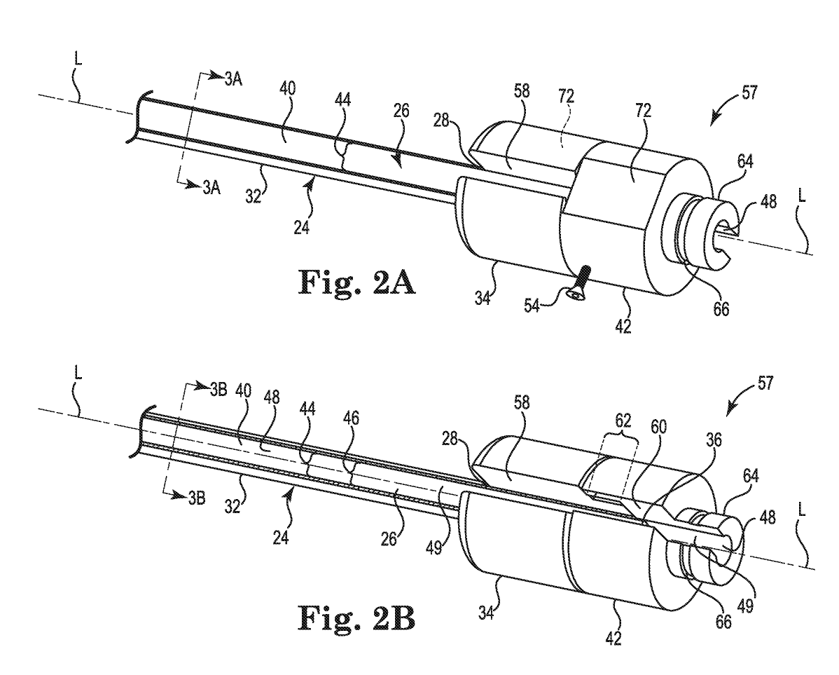

FIGS. 1A and 1B are perspective views of an exemplary tunneling device. FIG. 1B is a magnified view of a distal portion of the tunneling device. Together, FIGS. 1A and 1B show a tunneling device 10 including a tunneling rod 12 and an introducer sheath 14. The tunneling rod 12 extends from a proximal end 16 (hidden in FIG. 1A) to a distal end 18 and may include a tapered tip 20 at the distal end 18, and a rod handle 22 connected to the proximal end 16. The introducer sheath 14 includes an outer sheath 24 and an inner sheath 26. The outer sheath 24 is elongated and includes a proximal end 28, a distal end 30, and an outer sheath wall 32 extending from the proximal end 28 to the distal end 30. In some embodiments, the outer sheath 24 may further include an outer sheath handle 34 connected to the proximal end 28. The inner sheath 26 is elongated and includes a proximal end 36 (FIG. 2B), a distal end 38, and an inner sheath wall 40 extending from the proximal end 36 to the distal end 38. In some embodiments, the inner sheath 26 may further include an inner sheath handle 42 connected to the proximal end 36 (FIG. 2B) and disposed proximal of the inner sheath handle 42.

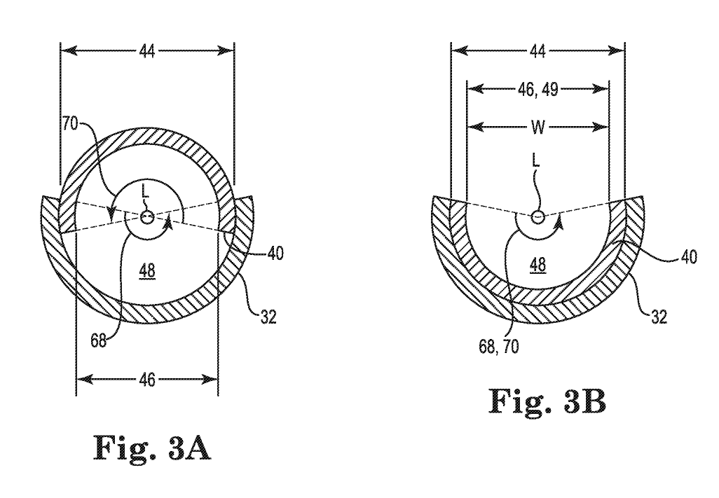

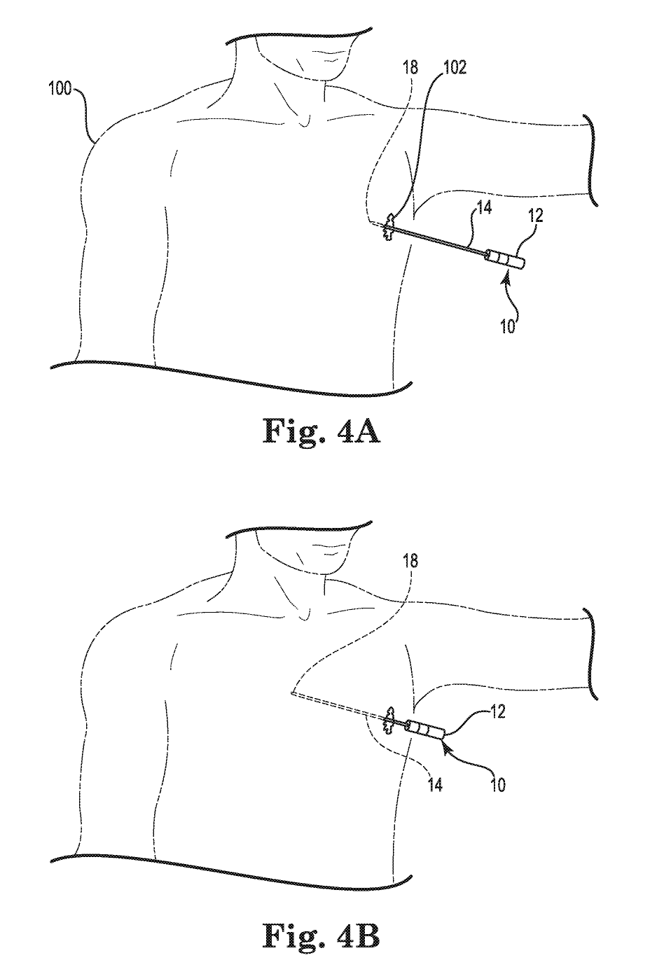

As shown in FIGS. 1A and 1B, the inner sheath 26 is coaxially disposed within the outer sheath 24, thus they share the same longitudinal axis L. The outer sheath wall 32 extends only partially circumferentially about the longitudinal axis L to define an outer sheath slot 44 extending from the proximal end 28 to the distal end 30. The inner sheath wall 40 also extends only partially circumferentially about the longitudinal axis L to define an inner sheath slot 46 (shown more clearly in FIG. 2B) extending from the proximal end 36 (FIG. 2B) to the distal end 38. The inner sheath 26 is rotatable relative to the outer sheath 24 to form an enclosed lumen 48 (shown more clearly in FIG. 3A) when the outer sheath slot 44 is not aligned with the inner sheath slot 46, as shown in FIGS. 1A and 1B, and to form an open slot 49 (FIG. 2B) along the entire length of the introducer sheath 14 when the outer sheath slot 44 is aligned with the inner sheath slot 46 (FIG. 2B). The tunneling rod 12 is removably inserted into the lumen 48 such that tapered tip 20 at the distal end 18 projects at least partially beyond the distal end 30 of the outer sheath 24 and the distal end 38 of the inner sheath 26 to define a distal end of the tunneling device 10. In some embodiments, the distal end 30 of the outer sheath wall 32 may include a chamfer 50, and the distal end 38 of the inner sheath wall 40 may include a chamfer 52, as shown in FIG. 1B. Together, the tapered tip 20 and the chamfers 50, 52 may ease a transit of the tunneling tool as it is driven to the desired location under the patient's skin, as described below in reference to FIGS. 4A and 4B.

In some embodiments, the introducer sheath 14 may further include a locking device 54 configured to selectively lock the outer sheath handle 34 to the inner sheath handle 42. Selectively locking the outer sheath handle 34 to the inner sheath handle 42 controls when the inner sheath 26 may rotate relative to the outer sheath 24. In some embodiments, the locking device 54 may be a set screw as shown in FIG. 1A.

In some embodiments, the outer sheath handle 34 and/or the inner sheath handle 42 may include a rotational stop (not shown) configured to prevent further rotation of the inner sheath 26 relative to the outer sheath 24 once the outer sheath slot 44 and the inner sheath slot 46 are aligned with each other.

In some embodiments, the tunneling rod 12 may further include a locking device 56 configured to selectively lock the rod handle 22 to the inner sheath handle 42. Unlocking the locking device 56 allows the rod handle 22 to be separated from the inner sheath handle 42 so that the tunneling rod 12 may be removed from the lumen 48 of the introducer sheath 14. In some embodiments, the locking device 56 may be a set screw as shown in FIG. 1A.

The tunneling rod 12 may be formed of a suitable rigid, biocompatible metal, for example, stainless steel, or a rigid, biocompatible polymer. The introducer sheath 14 may also be formed of a suitable rigid, biocompatible metal, for example, stainless steel, or a rigid, biocompatible polymer. However, in some embodiments in which the tunneling device 10 is curved, the introducer sheath 14 may be formed of a flexible, resilient polymer able to adapt to the curve of the tunneling rod 12 when the tunneling rod 12 is inserted into the lumen 48, and return to a straight configuration once the tunneling rod 12 is withdrawn from the introducer sheath 14.

FIGS. 2A-2B are perspective views of a proximal portion of the introducer sheath 14 with the tunneling rod 12 removed and showing a proximal end 57 of the introducer shaft 14. FIG. 2A shows the introducer sheath 14 with the outer sheath slot 44 not aligned with the inner sheath slot 46, thus forming the enclosed lumen 48 as described above. FIG. 2B shows the introducer sheath 14 with the outer sheath slot 44 aligned with the inner sheath slot 46 to form the open slot 49 along the entire length of the introducer sheath 14. As best shown in FIG. 2B, the outer sheath handle 34 includes an outer sheath handle slot 58 which is aligned with, and at least as wide as, the outer sheath slot 44. The inner sheath handle 42 includes an inner sheath handle slot 60 which is aligned with, and at least as wide as, the inner sheath slot 46. Thus, the outer sheath handle slot 58 and the inner sheath handle slot 60 extend the open slot 49 to the full length of the introducer sheath 14.

As shown in FIG. 2B, the outer sheath handle 34 and an inner sheath handle 42 may also include an overlap region 62 where portions of the outer sheath handle 34 and the inner sheath handle 42 overlap in the radial direction. The overlap region 62 may provide structure to support operation of locking device 54. The overlap region 62 may also provide structure to support a rotational stop (not shown).

As shown in FIG. 2A, the inner sheath handle 42 may include an overlap region 64 where portions of the inner sheath handle 42 and the rod handle 22 (FIG. 1A) overlap in the radial direction. The overlap region 64 may provide structure to support operation of the locking device 56, such as a channel 66 with which a set screw might engage to selectively secure the tunneling rod 12 to the introducer shaft 14.

As shown in FIG. 2A, the outer sheath handle 34 and the inner sheath handle 42 may each include a surface feature 72 (only surface feature 72 is visible in FIG. 2A). The surface features 72 are configured to provide a tactile and/or visible indication once the inner sheath handle 42 is rotated such that the inner sheath slot 46 aligns with the outer sheath slot 44 (FIG. 2B). In the embodiment shown in FIGS. 2A and 2B, the surface features 72 are flat surfaces disposed 180° from the outer sheath handle slot 58 and the inner sheath handle slot 60 providing both tactile and visual indications. In other embodiments, the surface features 72 may be surface markings providing only a visual indication.

In some embodiments, the outer sheath slot 44 and the inner sheath slot 46 are parallel to the longitudinal axis L, as shown in FIGS. 2A and 2B. In other embodiments, the outer sheath slot 44 and the inner sheath slot 46 may not be parallel to the longitudinal axis L, so long as they may be aligned to each other to form an open slot 49 along the entire length of the introducer sheath 14.

FIGS. 3A-3B are schematic cross-sections of the introducer sheath 14 showing the enclosed-lumen configuration and the open-slot configuration of FIGS. 2A and 2B. FIG. 3A shows the outer sheath slot 44 not aligned with the inner sheath slot 46, thus forming the enclosed lumen 48. FIG. 3B shows the outer sheath slot 44 aligned with the inner sheath slot 46, thus forming the open slot 49 along the entire length of the introducer sheath 14. As shown in FIGS. 3A and 3B, the outer sheath wall 32 extends circumferentially about the longitudinal axis L through an angle 68 to define the outer sheath slot 44. That is, the angle 68 is the extent to which the outer sheath wall 32 extends circumferentially about the longitudinal axis L. Similarly, the inner sheath wall 40 extends circumferentially about the longitudinal axis L through an angle 70 to define the inner sheath slot 46. The angle 68 is greater than 180° to ensure that the outer sheath 24 remains coaxial with the inner sheath 26. The angle 70 is greater than the difference between 360° and the angle 68 so that the lumen 48 may be fully enclosed when the outer sheath slot 44 is not aligned with the inner sheath slot 46. Both of the angles 68, 70 are less than 360° to ensure that the open slot 49 may be formed along the entire length of the introducer sheath 14. In some embodiments, the angle 68 may be at least 190° and no more than 200°, and the angle 70 may be at least 190° and no more than 200°.

In the embodiment shown in FIGS. 3A and 3B, the angle 68 and the angle 70 are about the same, with the width of the inner sheath slot 46 determining the width of the open slot 49 along at least a portion of the introducer sheath 14. In some embodiments, a width W of the inner sheath slot 46 in a direction perpendicular to the longitudinal axis L may range from about 0.125 inches (3.18 mm) to about 0.5 inches (12.7 mm). In other embodiments, the angles 68 and 70 may not be about the same. In some embodiments, the width of the outer sheath slot 44 may determine the width of the open slot 49 along at least a portion of the introducer sheath 14.

FIGS. 4A and 4B illustrate steps of implanting a lead or catheter within a patient 100 in accordance with disclosed embodiments. As shown in FIG. 4A, an incision 102 is made into the patient 100 at suitable location and a distal end of the tunneling device 10 is inserted into the incision and into the patient 100. The distal end of the tunneling device 10 includes the distal end 18 of the tunneling rod 12, the distal end 30 of the outer sheath 24, and the distal end 38 of the inner sheath 26 (FIG. 1B). During insertion, the inner sheath slot 46 is not aligned with the outer sheath slot 44, as shown in FIG. 3A. In some embodiments, the incision 102 is an opening through which the tunneling device 10 is inserted subcutaneously into the patient 100.

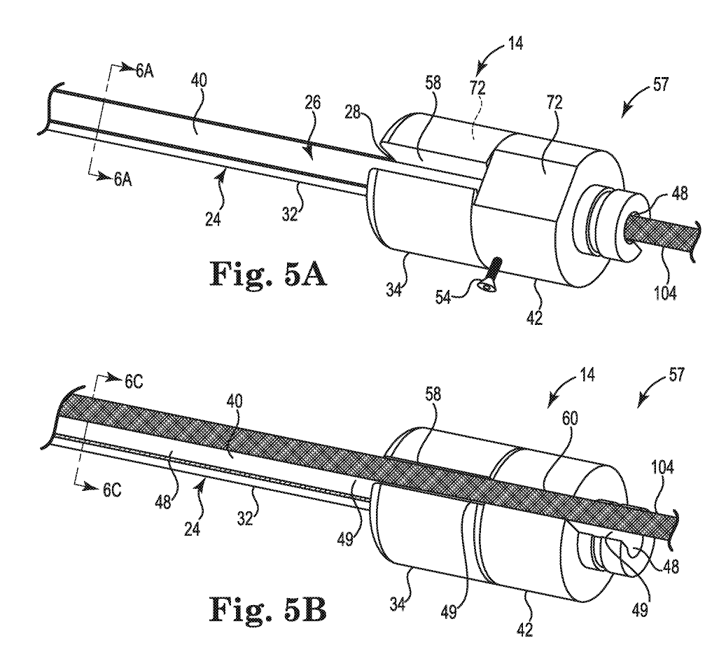

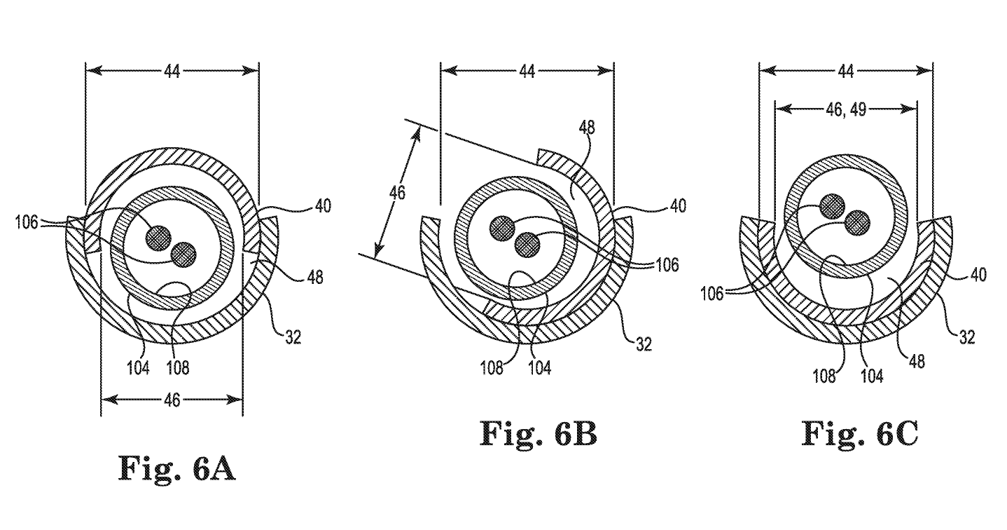

As shown in FIG. 4B, after the distal end of the tunneling device 10 is inserted into the incision 102, the distal end of the tunneling device 10 is driven to a location within the patient 100 corresponding to a desired placement of the lead or catheter to form a tunnel to the location. Once the desired location is reached, the tunneling rod 12 may be withdrawn from the proximal end 57 of the introducer sheath 14, leaving a tunnel to the location by way of the lumen 48 formed by the outer sheath 24 and the inner sheath 26 (FIG. 2A). Once the tunnel is formed, a lead or catheter (e.g., a lead 104 shown in FIGS. 5A-6C) is inserted into the lumen 48 of the introducer sheath 14 until the lead or catheter projects beyond the distal end of the introducer sheath 14.

FIGS. 5A and 5B are perspective views of a portion of the introducer sheath 14 illustrating additional steps of implanting a lead or catheter within the patient 100. FIG. 5A shows the introducer sheath 14 with a lead 104 inserted into lumen 48 as described above in reference to FIG. 4B. Once the lead 104 projects beyond the distal end of the introducer sheath 14 and is placed as desired, the inner sheath 26 is rotated relative to the outer sheath 26 until the inner sheath slot 46 and the outer sheath slot 44 are aligned to each other to form the open slot 49 along the entire length of the introducer sheath 14, as shown in FIG. 5B.

FIGS. 6A-6C are schematic cross-sections of the introducer sheath 14 further illustrating the additional steps shown in FIGS. 5A and 5B. FIG. 6A illustrates the configuration shown in FIG. 5A, in which the outer sheath slot 44 is not aligned with the inner sheath slot 46, thus forming the enclosed lumen 48. As shown in FIG. 6A, the outer sheath wall 32 and the inner sheath wall 40 together provide the enclosed lumen 48, containing the lead 104. The lead 104 may include a plurality of conductors 106 extending along a lead lumen 108. Considering FIGS. 5A and 6A together, rotating the inner sheath 26 relative to the outer sheath 24 may include unlocking the outer sheath handle 34 from the inner sheath handle 42 by loosening the locking device 54 (illustrated as a set screw). Once unlocked, the inner sheath handle 42 may be rotated relative to the outer sheath handle 34, to begin rotating the inner sheath wall 40 relative to the outer sheath wall 32, as shown in FIG. 6B. In FIG. 6B, the inner sheath slot 46 and the outer sheath slot 44 are still not aligned with each other and the lumen 48 is still sufficiently enclosed to contain the lead 104.

FIG. 6C shows the configuration shown in FIG. 5B, in which the outer sheath slot 44 is aligned with the inner sheath slot 46, thus forming the open slot 49. Once the open slot 49 is formed, the locking device 54 may be tightened to lock the outer sheath handle 34 to the inner sheath handle 42 to prevent further, unintended rotation of the inner sheath 26 relative to the outer sheath 24. As shown in FIG. 6C, the open slot 49 is sufficiently wide that the lead 104 may pass through the open slot 49. Thus, once the outer sheath slot 44 is aligned with the inner sheath slot 46, the introducer sheath 14 may be withdrawn from the patient 100 by angling the introducer sheath 14 slightly so that the lead 104 may pass through the open slot 49 as the introducer sheath 14 is withdrawn from the patient 100.

In some embodiments, once unlocked, the inner sheath handle 42 may be rotated relative to the outer sheath handle 34 until the rotational stop (not shown) corresponding to the alignment of the inner sheath slot 46 and the outer sheath slot 44 to each other is reached, before locking the inner sheath handle 42 to the outer sheath handle 34. Additionally or alternatively, alignment of the outer sheath slot 44 to the inner sheath slot 46 may be verified by tactile and/or visible indication of the surface features 72 before withdrawing the introducer sheath 14 from the patient 100.

The embodiments in reference to FIGS. 5A-6C, are illustrated with the lead 104. However, it is understood that the lead 104 is merely exemplary and embodiments may be used with leads of other designs as well as catheters.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

I claim:

1. An introducer sheath comprising:
an elongated outer sheath including a proximal end, a distal end, and an outer sheath wall extending from the proximal end to the distal end, the outer sheath wall extending circumferentially about a longitudinal axis of the outer sheath to an extent greater than 180° and less than 360° to define an outer sheath slot extending from the proximal end through the distal end, the outer sheath slot parallel to the longitudinal axis; and
an elongated inner sheath including a proximal end, a distal end, and an inner sheath wall extending from the proximal end to the distal end, the inner sheath wall extending circumferentially about a longitudinal axis of the inner sheath to an extent greater than the difference between 360° and the circumferential extent of the outer sheath wall, and less than 360° to define an inner sheath slot extending from the proximal end through the distal end, the inner sheath slot parallel to the longitudinal axis,
wherein the inner sheath is coaxially disposed within the outer sheath, the inner sheath rotatable relative to the outer sheath and configured to form an enclosed lumen when the outer sheath slot is not aligned with the inner sheath slot, and to form an open slot along the entire length of the introducer sheath when the outer sheath slot is aligned with the inner sheath slot.

2. The introducer sheath of claim 1, wherein the outer sheath wall extends circumferentially about the longitudinal axis of the outer sheath to an extent of at least 190° to no more than 200°, and the inner sheath wall extends circumferentially about the longitudinal axis of the inner sheath to an extent of at least 190° to no more than 200°.

3. The introducer sheath of claim 1, wherein a width of the inner sheath slot in a direction perpendicular to the longitudinal axis of the inner sheath ranges from about 0.125 inches (3.18 mm) to about 0.5 inches (12.7 mm).

4. The introducer sheath of claim 1, wherein the distal ends of each of the outer sheath wall and the inner sheath wall include a chamfer.

5. The introducer sheath of claim 1, further including:
an outer sheath handle connected to the proximal end of the outer sheath, the outer sheath handle including an outer sheath handle slot aligned with, and at least as wide as, the outer sheath slot; and
an inner sheath handle connected to the proximal end of the inner sheath, the inner sheath handle including an inner sheath handle slot aligned with, and at least as wide as, the inner sheath slot, wherein the inner sheath handle is rotatable relative to the outer sheath handle to rotate the inner sheath relative to the outer sheath.

6. The introducer sheath of claim 5, further including a locking device configured selectively lock the outer sheath handle to the inner sheath handle to selectively lock rotational positions of the outer sheath and the inner sheath with respect to each other.

7. The introducer sheath of claim 6, wherein the locking device is a set screw.

8. The introducer sheath of claim 5, wherein at least one of the outer sheath handle and the inner sheath handle include a rotational stop configured to prevent further rotation of the inner sheath relative to the outer sheath once the outer and inner sheath slots are aligned with each other.

9. The introducer sheath of claim 5, wherein the outer sheath handle and the inner sheath handle each include surface features configured to provide at least one of a visual indication and a tactile indication once the outer and inner sheath slots are aligned with each other.

10. A tunneling device for implanting a lead or catheter, the device comprising:
an introducer sheath including:
an elongated outer sheath including a proximal end, a distal end, and an outer sheath wall extending from the proximal end to the distal end, the outer sheath wall extending circumferentially about a longitudinal axis of the outer sheath to an extent greater than 180° and less than 360° to define an outer sheath slot extending from the proximal end through the distal end, the outer sheath slot parallel to the longitudinal axis; and
an elongated inner sheath including a proximal end, a distal end, and an inner sheath wall extending from the proximal end to the distal end, the inner sheath wall extending circumferentially about a longitudinal axis of the inner sheath to an extent greater than the difference between 360° and the circumferential extent of the outer sheath wall, and less than 360° to define an inner sheath slot extending from the proximal end through the distal end, the inner sheath slot parallel to the longitudinal axis, wherein the inner sheath is coaxially disposed within the outer sheath, the inner sheath rotatable relative to the outer sheath and configured to form an enclosed lumen when the outer sheath slot is not aligned with the inner sheath slot, and to form an open slot along the entire length of the introducer sheath when the outer sheath slot is aligned with the inner sheath slot; and
a tunneling rod removably inserted into the lumen of the introducer sheath, the tunneling rod extending from a proximal end to a distal end, the distal end including a tapered tip, the tapered tip projecting at least partially beyond the distal ends of the outer sheath and the inner sheath and defining a distal end of the tunneling device.

11. The tunneling device of claim 10, wherein the tunneling rod is curved along at least a portion of its length and the introducer sheath is formed of a material that is flexible and resilient.

12. The tunneling device of claim 10, wherein:
the introducer sheath further includes:
an outer sheath handle connected to the proximal end of the outer sheath, the outer sheath handle including an outer sheath handle slot aligned with, and at least as wide as, the outer sheath slot; and
an inner sheath handle connected to the proximal end of the inner sheath, the inner sheath handle including an inner sheath handle slot aligned with, and at least as wide as, the inner sheath slot, wherein the inner sheath handle is rotatable relative to the outer sheath handle to rotate the inner sheath relative to the outer sheath; and
the tunneling rod further includes a rod handle connected to the proximal end of the tunneling rod, the rod handle proximal to the inner sheath handle.

13. The tunneling device of claim 10, wherein the introducer sheath further includes a locking device configured selectively lock the outer sheath handle to the inner sheath handle to selectively lock rotational positions of the outer sheath and the inner sheath with respect to each other.

14. The tunneling device of claim 13, wherein the locking device is a set screw.

15. The tunneling device of claim 10, wherein at least one of the outer sheath handle and the inner sheath handle include a rotational stop configured to prevent further rotation of the inner sheath relative to the outer sheath once the outer and inner sheath slots are aligned with each other.

16. The tunneling device of claim 10, wherein the outer sheath handle and the inner sheath handle each include surface features configured to provide at least one of a visual indication and a tactile indication once the outer and inner sheath slots are aligned with each other.

17. The tunneling device of claim 12, wherein the tunneling rod further includes a locking device configured to selectively lock the rod handle to at last one of the outer sheath handle and the inner sheath handle.

18. A method for implanting a lead or catheter within a patient, the method comprising:
making an incision at a location of implantation;
inserting into the incision a distal end of a tunneling device including a tunneling rod and an introducer sheath, the introducer sheath including an outer sheath coaxially aligned with an inner sheath, the outer sheath including an outer sheath slot extending the length of the outer sheath, the inner sheath including an inner sheath slot extending the length of the inner sheath, the inner sheath slot not aligned with the outer sheath slot;
driving the distal end of the tunneling device to a location corresponding to the desired placement of the lead or catheter;
removing the tunneling rod from a proximal end of the introducer sheath;
inserting the lead or catheter into the introducer sheath until the lead or catheter projects beyond a distal end of the introducer sheath;
rotating the inner sheath relative to the outer sheath until the inner sheath slot and the outer sheath slot are aligned to each other to form an open slot along the entire length of the introducer sheath; and
withdrawing the introducer sheath from the patient, wherein the implanted lead or catheter passes through the open slot.

19. The method of claim 18, wherein the introducer sheath includes an outer sheath handle connected to a proximal end of the outer sheath, the outer sheath handle including an outer sheath handle slot aligned with, and at least as wide as, the outer sheath slot; and an inner sheath handle connected to a proximal end of the inner sheath, the inner sheath handle including an inner sheath handle slot aligned with, and at least as wide as, the inner sheath slot, wherein the inner sheath handle is rotatable relative to the outer sheath handle to rotate the inner sheath relative to the outer sheath, wherein rotating the inner sheath relative to the outer sheath includes:
unlocking the outer sheath handle from the inner sheath handle;
rotating the inner sheath handle relative to the outer sheath handle until a rotational stop is reached, the rotational stop corresponding to the alignment of the inner sheath slot and the outer sheath slot to each other; and
locking the outer sheath handle to the inner sheath handle.

20. The method of claim 19, wherein the outer sheath handle and the inner sheath handle each include surface features configured to provide at least one of a visual indication and a tactile indication once the outer and inner sheath slots are aligned with each other, the method further including:
verifying alignment of the outer and inner sheath slots with each other by at least one of visual and tactile indication of the surface features before withdrawing the introducer sheath from the patient.
